(19) United States
(12) Patent Application Publication
(10) Pub. No.: US 2025/0172501 A1
(43) Pub. Date: May 29, 2025

(54) MEASUREMENT CHIP, MEASURING DEVICE AND MEASURING METHOD

(71) Applicant: Furuno Electric Co., Ltd., Nishinomiya-City (JP)

(72) Inventors: Keiji TADA, Kobe (JP); Takeshi KAWAJIRI, Kawanishi (JP); Syoichiro KAJI, Osaka (JP)

(73) Assignee: Furuno Electric Co., Ltd., Nishinomiya-City (JP)

(21) Appl. No.: 19/039,781

(22) Filed: Jan. 29, 2025

Related U.S. Application Data
(63) Continuation of application No. PCT/JP2023/031645, filed on Aug. 30, 2023.

(30) Foreign Application Priority Data
Dec. 7, 2022 (JP) 2022-195511

(51) Int. Cl. G01N 21/77; G01N 21/41

(52) U.S. Cl. CPC G01N 21/7703; G01N 2021/4173; G01N 2021/7776

(57) ABSTRACT

Embodiments of the present disclosure provide a measurement chip. The measurement chip may include a propagation layer configured to allow light to propagate in a propagation direction, an introductory part configured to introduce the light into the propagation layer, an outgoing part configured to outgo the light from the propagation layer, and a coating layer configured to be formed on a surface of the propagation layer. A length of a formed region of the coating layer in the propagating direction is increased or decreased along a direction perpendicular to the propagating direction. The length of the formed region of the coating layer is modifiable using a ligand that reacts with an analyte on the surface of the propagation layer at least in an exposed area that is exposed from the coating layer.

### CROSS-REFERENCE TO RELATED APPLICATION(S)

This application is a continuation application of PCT International Application No. PCT/JP2023/031645, which was filed on Aug. 30, 2023, and which claims priority to Japanese Patent Application No. JP2022-195511 filed on Dec. 7, 2022, the entire disclosures of each of which are herein incorporated by reference for all purposes.

### TECHNICAL FIELD

The present disclosure relates generally to biosensors, and specifically to measurement chips, measuring devices and measurement methods for measuring a change in a pattern of light.

### BACKGROUND

Biosensors are analytical devices that combine a biological component with a physicochemical detector to detect and measure presence of specific biological or chemical substances. These devices are designed to convert biological responses into measurable signals, enabling quantification of various analytes in a sample. Biosensors find applications in diverse fields, including medical diagnostics, environmental monitoring, food safety, and more.

In an example, a biosensor may be used to analyze interactions between chemical substances (e.g., biomolecules). Types of biosensors may include, for example, optical waveguides-based biosensors, and t surface plasmon resonance-based biosensors, and Mach-Zehnder interference-based biosensors.

Conventionally, optical waveguides-based biosensors (hereafter also referred to as, optical waveguide type measurement chip or measurement chip) may use an optical waveguide to detect a substance (namely, an analyte) and a reactant (namely, a ligand) that reacts with the analyte that may be formed on a surface of a propagation layer where light propagates. In optical waveguide type measurement chips, presence or absence of the analyte and a concentration of the analyte may be estimated based on a change in a light pattern derived from the propagation layer, using an amount of difference in phase change of the light propagating in the propagation layer between a region where the ligand is immobilized and a region where the ligand is not immobilized.

Further, in certain other conventional cases, an optical waveguide type biosensor may use a coloring agent and detects an intensity change of the propagating light due to absorption and scattering of light.

However, conventional biosensors, specifically, the optical waveguide type biosensors fail to measure accurately and reliably. Therefore, optical waveguide-type measurement chips or biosensors require performance improvements in terms of measurement reproducibility and measurement reliability.

### SUMMARY

A first aspect of the present disclosure relates to a measurement chip. The measurement chip includes a propagation layer configured to allow light to propagate in a propagating direction. The measurement chip may further include an introductory part [an introductory coupler] configured to introduce the light into the propagation layer. The measurement chip may further include an outgoing part [an outgoing coupler] configured to outgo the light from the propagation layer. The measurement chip may further include a coating layer configured to be formed on a surface of the propagation layer. The coating layer increases or decreases a length of the propagating direction in a formed region of a coating, along a direction perpendicular to the propagating direction. Moreover, a ligand reacts with an analyte on the surface of the propagation layer at least in an exposed area exposed from the coating layer.

According to an embodiment, the measurement chip may further include a ligand layer formed by modifying the ligand on the surface of the propagation layer in the exposed area.

According to an embodiment, a first refractive index of the coating layer is smaller than a second refractive index of the propagation layer and greater than a third refractive index of the ligand layer.

According to an embodiment, the coating layer is configured to be formed between the introductory part and the outgoing part of the measurement chip.

According to an embodiment, the coating layer is configured to be formed on at least one of: the introductory part, or the outgoing part of the measurement chip.

According to an embodiment, the coating layer is further configured to continuously increase or decrease the length of the formed region of the coating in the propagation direction, that along the direction perpendicular to the propagating direction.

According to an embodiment, the coating layer is further configured to linearly increase or decrease the length of the formed region of the coating in the propagation direction, along the direction perpendicular to the propagating direction.

According to an embodiment, the coating layer has a thickness equal to or greater than a decay length of evanescent light penetrating from the surface of the propagation layer towards a medium on a side of the coating layer.

According to an embodiment, the coating layer is formed using silicon dioxide.

According to an embodiment, the coating layer is formed using a mixture of silicon dioxide and a metal oxide.

According to an embodiment, the coating layer is formed using a mixture of silicon dioxide and aluminum oxide (Al₂O₃).

According to an embodiment, the measurement chip may further include a characteristic adjustment film arranged on a surface of the coating layer.

According to an embodiment, the characteristic adjustment film is formed using a metal oxide.

According to an embodiment, a phase distribution of the light changes due to a change in a refractive index around the propagation layer caused by a reaction between the analyte with the ligand.

In another aspect, a measuring device is provided. The measuring device includes a measurement chip. The measurement chip comprises a propagation layer configured to allow light to propagate in a propagating direction, an introductory part configured to introduce the light into the propagation layer, an outgoing part configured to outgo the light from the propagation layer, and a coating layer configured to be formed on a surface of the propagation layer. The coating layer increases or decreases a length of the propagating direction in a formed region of a coating, along a direction perpendicular to the propagating direction. Moreover, a ligand reacts with an analyte on the surface of the propagation layer at least in an exposed area exposed from the coating layer. The measuring device may further include a light source configured to introduce the light to the introductory part of the measurement chip. The measuring device further includes a photodetector configured to receive the light outgone from the outgoing part of the measurement chip. The measuring device further includes a control unit (which is also referred to as a processing circuitry or a controller) configured to analyze a change in a pattern of the light received by the photodetector that changes based on the reaction between the analyte and the ligand of the measurement chip.

According to an embodiment, the control unit is further configured to analyze a change in the propagating direction of the light.

In yet another aspect, a measurement method is provided. The measurement method includes introducing light into a propagation layer. The measurement method further includes causing the light to totally reflect in the propagation layer having a surface on which a ligand reacting to an analyte is formed in an exposed area from a coating layer formed on the surface of the propagation layer. The measurement method further includes deriving the light from the propagation layer. The coating layer increases or decreases a length of the propagating direction in a formed region of a coating, along a direction perpendicular to the propagating direction.

According to an embodiment, the measurement method further includes analyzing a change in a pattern of the light derived from the propagation layer. The change in the pattern is caused due to a reaction between the analyte and the ligand of the measurement chip.

According to an embodiment, the measurement method further includes analyzing a change in a traveling direction of the light derived from the propagation layer.

According to an embodiment, a first refractive index of the coating layer is smaller than a second refractive index of the propagation layer and greater than a third refractive index of a ligand layer that is formed by modifying the surface of the propagation layer in the exposed area using the ligand.

### BRIEF DESCRIPTION OF DRAWINGS

The embodiments of the present disclosure is illustrated by way of example and not by way of limitation in the figures of the accompanying drawings, in which the like reference numerals indicate like elements and in which:

FIGS. 1(A)-1(C) are diagrams showing different views of a schematic structure of a measurement chip, in accordance with various embodiments of the present disclosure;

FIGS. 2(A) and 2(B) are diagrams showing a top view and a cross-sectional view, respectively, of the schematic structure of the measurement chip, in accordance with different embodiments of the present disclosure;

FIG. 3 is a schematic diagram of a configuration of a measuring device that includes the measurement chip, in accordance with an embodiment of the present disclosure;

FIGS. 4(A)-4(C) are diagrams illustrating signals measured by the measurement chip, in accordance with an embodiment of the present disclosure;

FIG. 5 is a flowchart illustrating a measurement method, in accordance with an embodiment of the present disclosure;

FIGS. 6(A)-6(E) are diagrams showing variations in a planar shape of a coating layer in the measurement chip, in accordance with an embodiment of the present disclosure;

FIG. 7 is a graph showing results of a performance evaluation of the measurement chip, in accordance with an embodiment of the present disclosure;

FIGS. 8(A) and 8(B) illustrates schematic structure of conventional measurement chip, in accordance with an embodiment; and

FIGS. 9(A)-9(C) are diagrams showing different methods of forming a ligand layer in a specific planar shape in a conventional measurement chip, in accordance with an embodiment.

The purpose of the present invention is to provide optical waveguide type measurement chips, measuring devices, and measuring methods that further improve measurement reproducibility and measurement reliability, and further reduce the cost of the fabrication process.

### DETAILED DESCRIPTION

Embodiments of the disclosure will be described in detail below with reference to the accompanying drawings. It should be noted that in the following descriptions and drawings, the same symbols refer to the same or similar components, and thus duplicate descriptions of the same or similar components are omitted.

In the following description, for purposes of explanation, numerous specific details are set forth in order to provide a thorough understanding of the present disclosure. It will be apparent, however, to one skilled in the art that the present disclosure may be practiced without these specific details. In other instances, systems and methods are shown in block diagram form only in order to avoid obscuring the present disclosure.

Some embodiments of the present disclosure will now be described more fully hereinafter with reference to the accompanying drawings, in which some, but not all, embodiments of the disclosure are shown. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout. Also, reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described which may be exhibited by some embodiments and not by others. Similarly, various requirements are described which may be requirements for some embodiments but not for other embodiments.

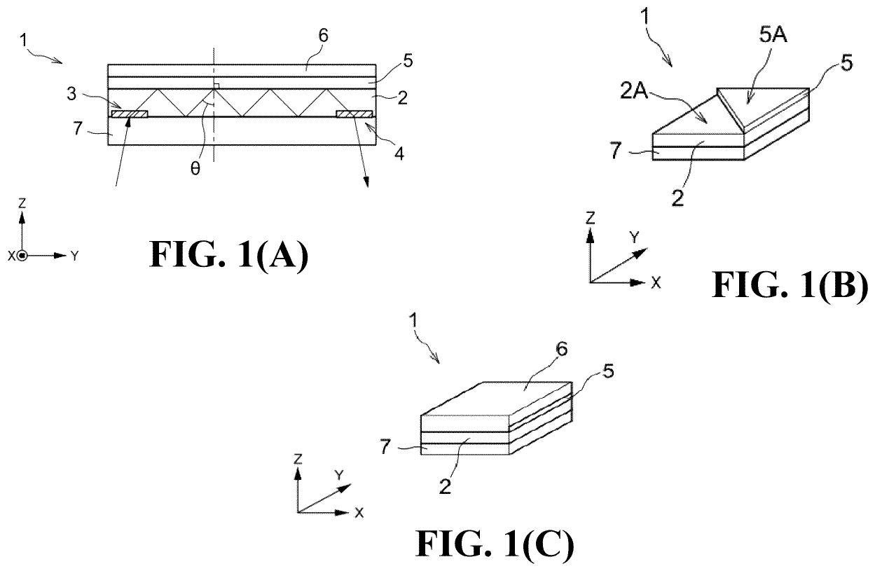

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect. Turning now to FIGS. 1-FIGS. 9, a brief description concerning the various components of the present disclosure will now be briefly discussed. Reference will be made to the figures showing various embodiments of a measurement chip comprising a coating layer that is modified due to a reaction between a ligand and an analyte.

### Measurement Chip

FIGS. 1(A)-1(C) are diagrams showing different views of a schematic structure of a measurement chip 1, in accordance with various embodiments of the present disclosure. For example, the FIG. 1(A) shows a side view of the measurement chip 1, according to an embodiment. Moreover, FIG. 1(B) and FIG. 1(C) show a side view of the measurement chip 1, according to an embodiment.

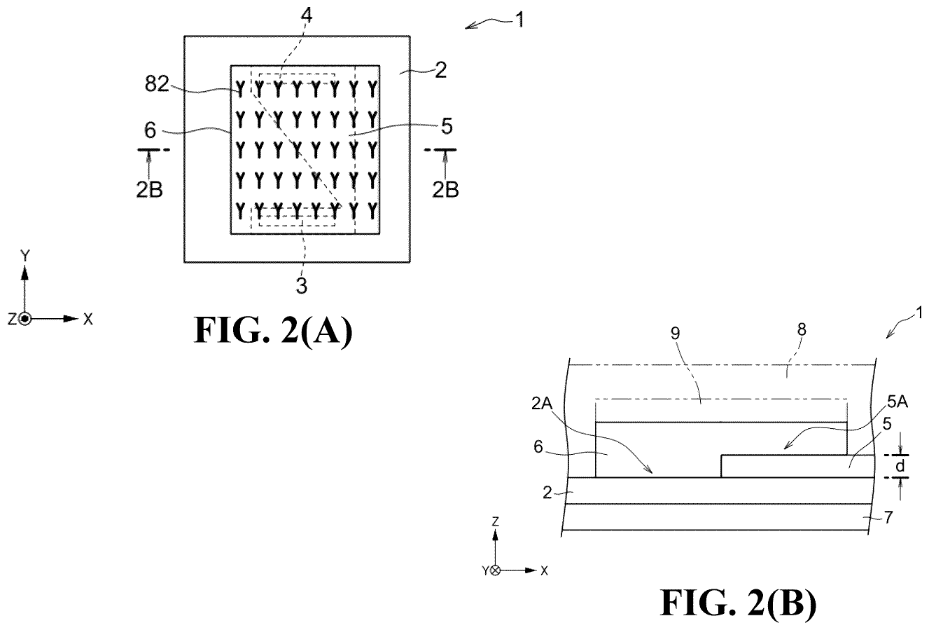

FIG. 2(A) and FIG. 2(B) shows the schematic structure of the measurement chip 1, according to an embodiment. For example, FIG. 2(A) shows a top view of the measurement chip 1, according to an embodiment. FIG. 2(B) shows a cross-sectional view of the measurement chip 1, according to an embodiment.

Referring to FIGS. 1(A)-1(C) and FIGS. 2(A) and 2(B), a top surface direction (thickness direction) of the measurement chip 1 is defined in a Z-axis direction, a propagating direction of light in the measurement chip 1 is defined in a Y-axis direction, and a vertical direction that is perpendicular to the propagating direction is defined in an X-axis direction. In addition, a surface of the measurement chip 1 indicates either a top surface or a bottom surface, and both surfaces indicate both the top surface and the bottom surface.

According to some embodiments of the present disclosure, some configurations are omitted for the sake of ease of drawing, and the dimension ratios of the configurations shown are also emphasized.

The measurement chip 1 comprises a propagation layer 2. The propagation layer 2 is configured to allow light to propagate in a propagating direction. Moreover, the measurement chips 1 comprises an introductory part [an introductory coupler] 3 and an outgoing part [an outgoing coupler] 4. In an example, the introductory part 3 is configured to introduce the light into the propagation layer 2. Moreover, the outgoing part 4 is configured to outgo the light from the propagation layer 2.

In FIG. 2(A) and FIG. 2(B), the propagation layer 2 is drawn along the X-axis and the Y-axis directions in the figure in accordance with a manner in which the measurement chip 1 may be used for measurement.

According to an embodiment of the present disclosure, an overview of the measurement chip 1 will be described with reference to FIGS. 1(A)-1(C).

FIGS. 1(A)-1(C) are diagrams showing the side view and the perspective view of the schematic structure of the measurement chip 1, in accordance with an embodiment. As shown in FIG. 1(A) and FIG. 1(B), the measurement chip 1 may include the propagation layer 2 through which light propagates, the introductory part 3 for introducing light into the propagation layer 2, the outgoing part 4 for deriving light from the propagation layer 2, and a coating layer 5 formed on a surface of the propagation layer 2. In an example, the coating layer 5 is formed in a specific planar shape on the surface of the propagation layer 2.

In an example, the measurement chip 1 is designed to have, on the surface of the propagation layer 2 a ligand (for example, antibodies) that may react with analyte (for example, antigen) in an object to be measured (for example, a specimen). for example, such reaction between the ligand and the analyte may form a ligand layer 6 on the surface of the propagation layer 2.

Referring to FIG. 1(B) and FIG. 1(C), the ligand layer 6 is uniformly formed on a surface 2A of the propagation layer 2 and a surface 5A of the coating layer 5. It may be noted that the propagation layer 2 lies below the coating layer 5, i.e., a formed region of a coating is formed on the propagation layer 2, such that the formed region of the coating forms the coating layer 5.

For example, the coating layer 5 is formed on the surface of the propagation layer 2 in a specific planar shape. In an embodiment, the ligand layer 6 may be formed on an entirety of the surface 2A of the propagation layer 2 and the surface 5A of the coating layer 5. In another embodiment, the ligand layer 6 may be formed at least on an area (referred to as an exposed area) on the surface 2A of the propagation layer 2. The exposed area is exposed from the coating layer 5.

In an example, a reaction (or a binding) between the ligand with the analyte, bulk effects, and non-specific adsorption may occur on an entire surface of the ligand layer 6. Further, the coating layer 5 may be formed with a thickness that attenuates evanescent light such that evanescent light penetrating from an inside of the propagation layer 2 and moving towards the ligand in the ligand layer 6 may be attenuated and make the evanescent light difficult to sense.

To overcome the above-mentioned problem, the measurement chip 1 may be utilized to sense the reaction of the ligand with the analyte, bulk effect, and non-specific adsorption only on the surface 2A of the propagation layer 2 in the exposed area exposed from the coating layer 5. Thus, the reaction of the ligand with the analyte, bulk effect, and non-specific adsorption may be sensed only on the surface 2A of the propagation layer 2 in the exposed area exposed from the coating layer 5, as the coating layer 5 is formed with a thickness that attenuates evanescent light, even if the ligand layer 6 is formed on the entire surface 2A and 5A of the propagation layer 2 and the coating layer 5. In an example, due to the reaction (binding) between the analyte and the ligand, a refractive index of the propagation layer 2 and the coating layer 5 may change. As a result, a phase distribution in the X-axis direction and the Y-axis direction of the propagating direction of the light changes on the surface 2A of the propagation layer 2 where the ligand layer 6 is formed. Thus, the measurement chip 1 may be utilized as a measurement chip for estimating presence or absence or concentration of the analyte.

According to an embodiment, the coating layer 5 is formed in a specific planar shape on the surface 2A of the propagation layer 2 in the measurement chip 1. Thus, even if the ligand layer 6 is formed uniformly on the entire surfaces 2A and 5A of the propagation layer 2 and the coating layer 5, respectively, the ligand layer 6 is formed on the surface 2A of the propagation layer 2 in the exposed area exposed from the coating layer 5 in a specific planar shape. A pattern of the specific planar shape is inverted from the planar shape of the coating layer 5. Further, the formation of the coating layer 5 may be carried out by, for example, vapor deposition regardless of a wet process. To this end, measurement reproducibility may be improved and a cost of fabrication of the measurement chip 1 may be reduced. Therefore, the measurement chip 1 of the present disclosure may improve measurement reproducibility and measurement accuracy of the optical waveguide-based measurement chips.

According to some embodiments, each part of the measurement chip 1 may be described with reference to FIG. 1 and FIG. 2.

In an example embodiment, the propagation layer 2 is flat. For example, the light may be introduced into the propagation layer 2 from the introductory part 3, such that the light is totally reflected on an upper and a lower surface of the propagation layer 2. Further, the light may be derived out from the outgoing part 4.

Pursuant to present disclosure, a deposited film (having a refractive index of, for example, 2.07, approximately, depending on a wavelength of the light) may be used in the propagation layer 2. The deposited film may be made of, for example, metal oxides, such as titanium oxide (TiO₂) and tantalum oxide (Ta₂O₅). In an embodiment, materials for manufacturing the propagation layer 2 may include, for example, a dielectric, such as acrylic resin, glass, polyvinyl alcohol, polyvinyl chloride, silicone resin or polystyrene in addition to the metal oxides. Further, a thickness of the propagation layer 2 may be varied, for example, in the Z-axis direction, and a length of the propagation layer 2 may be varied, for example, in the X-axis direction and the Y-axis direction. For example, the thickness may vary from about 50 nanometers (nm) to about 100 nm. Moreover, a length of the propagation layer 2 in the Y-axis direction may be, for example, 4 millimeters (mm); and the length of the propagation layer 2 in the X-axis direction may be in a range of, for example, 270 micrometers (μm) to 600 μm. In an example, use of the dielectric such as, acrylic resin, glass, polyvinyl alcohol, polyvinyl chloride, silicone resin or polystyrene as a material of the propagation layer 2 may provide a capability to the propagation layer 2 itself to function as a substrate. Therefore, the substrate 7 may be omitted. This reduces cost of fabrication of the measurement chip 1.

In another embodiment, the introductory part 3 and the outgoing part 4 may be provided in the propagation layer 2. Further, a diffraction grating may be used for the introductory part 3 and the outgoing part 4. In an example, the diffraction grating may be fabricated using a Nano-imprint method. In addition to the diffraction grating, the introductory part 3 and the outgoing part 4 may be fabricated, for example, using a prism. In an embodiment, the introductory part 3 and the outgoing part 4 may be provided on a lower surface of the propagation layer 2. However, in some embodiment, the introductory part 3 and the outgoing part 4 may be provided at an upper surface of the propagation layer 2 instead of the lower surface.

Further, the coating layer 5 may be formed in the specific planar shape on the surface 2A of the propagation layer 2. The coating layer 5 may need to be formed at least between the introductory part 3 and the outgoing part 4 of the measurement chip 1. In some embodiments, as illustrated in FIG. 1(A) and FIG. 2(A), the coating layer 5 may be formed on the introductory part 3 and the outgoing part 4 of the measurement chip 1. Further, in some embodiment, the coating layer 5 may be formed on either of the introductory part 3 or the outgoing part 4.

In an example, the specific planar shape of the coating layer 5 may be a length in the exposed area where the coating is formed in the coating layer 5. Further, the specific planar shape of the coating layer 5 is a shape in which the length of the formed region of the coating, that extends along the propagating direction (Y-axis direction) of the light, increases or decreases along the direction perpendicular to the propagating direction (X-axis direction). It may be noted that a part of the specific planar shape of the coating layer 5 may also include the part that increases or decreases, or a whole of the specific planar shape may increase or decrease. To include the part that increases or decreases in the part of the specific planar shape means that the specific planar shape is, for example, a parallelogram or a trapezoid. For example, the parallelogram or the trapezoid includes an increasing or a decreasing part in the part of the shape and also has a fixed length. Increasing or decreasing in the whole of a particular planar shape refers to a particular planar shape being, for example, a right angled-triangle. The right-angled triangle may include such increasing or decreasing parts throughout the shape. For an example, the specific planar shape may be a right triangle as depicted in FIG. 1(B).

In an exemplary embodiment, the coating layer 5 may have a thickness equal to or greater than a bleeding length of evanescent light penetrating from the surface 2A of the propagation layer 2 toward a medium on a side of the coating layer 5. For example, the bleeding length of evanescent light may refers to a distance over which the evanescent light decays and loses it's energy.

In an example, a wavelength of the evanescent light is about 520 nm. Subsequently, a thickness of the coating layer 5 may be equal to or greater than the bleeding length of the evanescent light. For example, the coating layer 5 may have a thickness (referred as ‘d’) of about 132 nm or greater, and more preferably a thickness of d may be of about 250 nm or greater. Theoretically, a first refractive index of the coating layer 5 is lower than a second refractive index of the propagation layer 2. Further, the first refractive index of the coating layer 5 may be equal or substantially equal to a third refractive index of the ligand layer 6.

It may be noted, a transparent material with high stability may have a higher refractive index than the third refractive index of the ligand layer 6. Therefore considering stability, a material with a lower refractive index of the coating layer 5 than that of the propagation layer 2 and higher than that of the ligand layer 6 may be optimal for the measurement chip 1.

In an example, in a case where silicon dioxide is used for making the coating layer 5, a vapor-deposited film consisting mainly of the metal oxides may be used for manufacturing of the propagation layer 2. Further, the thickness of the coating layer 5 is specifically illustrated based on a calculation of a seeping distance of the evanescent light. The seeping distance of the evanescent light may refers to a length at which an amplitude of the light is 1/e (intensity is 1/e²). The e (energy) may refer to energy of a single photon of the light. Further, the seeping (sometimes referred as ‘leaching’) distance of the evanescent light may be calculated from the first refractive index of the coating layer 5, the second refractive index of the propagation layer 2, the wavelength of light propagating into the propagation layer 2 and propagation angle of the light propagating through the propagation layer 2 (also referred as ‘Φ’). Here, the propagation angle, Φ, is an angle between an axis perpendicular to an interface between the propagation layer 2 and the coating layer 5 (e.g., Z axis in FIG. 1) and a direction in which the light travels in the propagation layer 2 (i.e., the propagating direction).

In an exemplary scenario, the second refractive index of the propagation layer 2 is 2.037, the first refractive index of the coating layer 5 formed on it may be 1.46, the wavelength of the light in vacuum is 520 nm, and the propagation angle, Φ, of the light propagating through the propagation layer 2 is 51.3°. In such a case, the leaching distance of the evanescent light is calculated to be 132.0 nm. Since, a region within a length/distance of the leaching distance may become a light absorption region, therefore a film thickness of the coating layer 5 may be more than or equal to 132.0 nm. In another exemplary scenario, the second refractive index of the propagation layer 2 is 1.987, the first refractive index of the coating layer 5 is 1.45, the wavelength of the light in vacuum is 810 nm, and the propagation angle, Φ, of the light propagating through the propagation layer 2 is 52.8°. In such a case, the leaching distance of evanescent light is calculated to be 203.6 nm. Subsequently, a region within this distance may become the light absorption region, and the film thickness of the coating layer 5 may be greater than or equal to 203.6 nm.

According to another embodiment, silicon dioxide (having the refractive index of about 1.47) may be used for making the coating layer 5. Silicon dioxide can be formed on the surface 2A of the propagation layer 2 by vapor deposition. In an embodiment, a metal mask may be used to form the planar shape of the coating layer 5 into a right-angled triangle as illustrated in FIG. 1(B). The metal mask with a right triangle hole, for example, having the shape of the coating layer 5 may be made, and the area other than the coating layer 5 may be masked with the metal mask to form the coating layer 5 into a right triangle during deposition. In addition to that, a rise in temperature during the deposition is small and the adhesion to the propagation layer 2 is high, resulting in less peeling in a salt solution, thereby making silicon dioxide a suitable deposition material for the coating layer 5. In addition to silicon dioxide used as a material for the coating layer 5, a metal oxide such as aluminum oxide (Al₂O₃), a fluorine-based low refractive index material (refractive index of about 1.33-1.38), or a mixture of these materials may also be used for making the coating layer 5. In an embodiment, use of the mixture of silicon dioxide and the metal oxide (such as Al₂O₃) whose main component is silicon dioxide may be used for making the coating layer 5. For example, magnesium fluoride (MgF₂) or thiolite (R) (Na₅Al₃F₁₄) may be used as a fluorine-based low refractive index material for the coating layer 5.

In an example embodiment, a characteristic adjustment film (not shown in figures) may be further formed on the surface 5A of the coating layer 5. The characteristic adjustment film may be formed using, for example, the metal oxides described above. By further coating and forming the characteristic adjustment film on the surface 5A of the coating layer 5, it may be possible to bring the characteristics of the exposed area and the coating layer 5 closer together and to further apply an equivalent surface treatment. This may improve measurement stability and measurement accuracy of the measurement chip 1.

In an embodiment, the ligand layer 6 may be formed on the surface 2A of the propagation layer 2. In this regard, a ligand may be a substance that specifically reacts or binds with an analyte. The analyte may be a detected substance in an object, such as a specimen, that needs to be measured. Further, the ligand layer 6 may be formed uniformly on the surfaces 2A and 5A of the propagation layer 2 and the coating layer 5, respectively. Alternatively, the ligand layer 6 may also be formed only on the surface 5A of the coating layer 5. However, the ligand layer 6 needs to be formed at least on the surface 2A of the propagation layer 2 in the exposed area exposed from the coating layer 5, i.e., in the exposed area on the propagation layer 2 and the surface 5A of the coating layer 5. In an example, the planar shape of the coating layer 5 is a right-angled triangle. Furthermore, between the introductory part 3 and the outgoing part 4, the planar shape of the ligand layer 6 in an area formed on the surface 2A of the propagation layer 2 in the exposed area exposed from the coating layer 5 is also a right-angled triangle. Here, the refractive index of the ligand layer 6 may be, for example, about 1.33.

Thus, on the surface 2A of the propagation layer 2 in the exposed area exposed from the coating layer 5, the ligand layer 6 is formed in the specific planar shape. The pattern of the specific planar shape is inverted from the specific planar shape of the coating layer 5. In the ligand layer 6, a content of the ligand in the propagating direction of light (e.g., Y-axis direction) may change monotonically along the direction perpendicular to the propagating direction (X-axis direction) in a formed region of coating located on the surface 2A of the propagation layer 2. As a result, a phase distribution in the X-axis direction of the light propagating in the Y-axis direction may change on the surface 2A of the propagation layer 2 on which the ligand layer 6 is formed. The phase distribution may change based on a change in the refractive index caused by the reaction (or binding) between the analyte and the ligand. Further, a content of the ligand may be calculated by multiplying a ligand content density per unit length in the light propagating direction by a length of the ligand layer 6 along the light propagating direction.

In one embodiment, a transparent substrate 7 having an arbitrary configuration may be provided on a lower surface of the propagation layer 2. For example, glass (having refractive index about 1.47-1.48) may be used for making the substrate 7. In another embodiment, the measurement chip 1 may further include an intermediate layer, such as a fluororesin, between the lower surface of the propagation layer 2 and the transparent substrate 7.

Referring to FIGS. 2(A) and 2(B), a top view and a cross-sectional of the schematic structure of the measurement chip 1 is shown. According to one embodiment, a mode in which the measurement chip 1 may be used for measurement will be described with reference to FIGS. 2(A) and 2(B).

At a time of use of the measurement chip 1 for measurement, a ligand 82 may be modified on the surface of the propagation layer 2 to form the ligand layer 6 on the surface 2A of the propagation layer 2. A base material 8 having a concave cross section, for example, may be provided on the upper surface of the measurement chip 1 to cover the propagation layer 2 on which the ligand layer 6 is formed. Further, a flow channel 9 may be provided between the base material 8 and the ligand layer 6, as shown by a dashed line in the cross section of FIG. 2(B).

In operation, a solution of an object or a specimen to be measured flows into the flow channel 9. Further, the analyte contained in the object may be measured using the measurement chip 1.

According to one embodiment, the light introduced into the propagation layer 2 through the introductory part 3 may propagate in the Y-axis direction in the propagation layer 2. Further, the introduced light may be derived out from the propagation layer 2 through the outgoing part 4. While propagating in the Y-axis direction in the propagation layer 2, the light may be affected by the second refractive index of the propagation layer 2. Further, the affected light may change due to a reaction between the analyte in the solution of the object (that is to be measured) flowing in the flow channel 9 and the ligand 82 in the ligand layer 6. In the measurement chip 1, the ligand layer 6 may be formed on the surface 2A of the propagation layer 2 such that a length of the ligand layer 6 in the light propagating direction (Y-axis direction) increases or decreases along the direction perpendicular to the propagating direction (X-axis direction). As a result, the phase distribution in the X-axis direction changes due to the influence of the change in the refractive index of the light propagating in the Y-axis direction on the surface 2A of the propagation layer 2 where the ligand layer 6 is formed.

FIG. 2(B) is a cross-sectional view of the measurement chip 1 along an axis 2B shown in the FIG. 2(A). In the cross-sectional view of FIG. 2(B), the reaction between the ligand 82 and the analyte, bulk effect and non-specific adsorption may be sensed on the surface 2A of the propagation layer 2 where the ligand layer 6 is formed (for example, on the left side of the FIG. 2(B)).

### Measuring Device

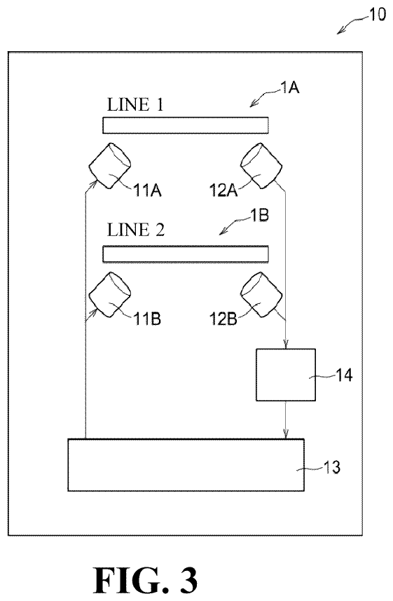

FIG. 3 is a schematic diagram of a configuration of a measuring device 10 that includes the measurement chip 1, in accordance with an embodiment. The measuring device 10 may include a first light source 11A and a second light source 11B (collectively referred as light sources 11) that may emit light that propagates through the introductory part 3 of the measurement chip 1, a first photodetector 12A and a second photodetector 12B (collectively referred as photodetectors 12) that may receive light derived from the introductory part 4 of the measurement chip 1, and a control unit (The measuring device further includes a control unit (which is also referred to as a processing circuitry or a controller) 13 that may analyze changes in a pattern (such as, intensity distribution) of the light received by the photo detectors 12. The pattern of the light received by the photodetectors 12 may change based on a contact between the solution of the object to be measured and the measurement chip 1 causing the reaction between the ligand 82 and the analyte of the object. Further, the measuring device 10 may also include a measuring unit (a light intensity sensor) 14 that may acquire intensity information of the light received by each light receiving element of the photo detectors 12.

The control unit 13 and the measuring unit 14 may be configured in hardware using, for example, a dedicated integrated circuit (IC), or implemented in software using an information processing device such as a general-purpose computer, a smartphone, or a tablet terminal. For example, the control unit 13 and the measuring unit 14 may be a computer including a CPU, a RAM, a ROM, a nonvolatile memory, an input/output interface, and the like. For example, the CPU of the processing circuitry 10 executes information processing according to a program loaded from a ROM or a nonvolatile memory into a RAM.

In one example, the measuring device 10 may include two sets of the measurement chip 1 (depicted as measurement chip 1A and measurement chip 1B), the light sources 11, at least one of the photo detectors 12. and the measuring device 10. The measuring device 10 may acquire two lines of measurement signals, for example, a first signal from a region where the ligand layer 6 is formed (line 1), and a second signal from a region where the ligand layer 6 is not formed (line 2). In the measurement chip 1A for the line 1, the ligand layer 6 may be formed uniformly on the surface 2A of the propagation layer 2 and the coating layer 5. Further, in the measurement chip 1B for the line 2, the ligand layer 6 may not be formed. The second signal of the line 2 may be described as a reference signal for subtracting the effects of bulk effects and non-specific adsorption from the first signal of line 1.

In another embodiment, the measuring device 10 may have one measurement chip 1, at least one of the light sources 11, and at least one of the photo detectors 12. In this case, two measurement lines, i.e., two optical waveguide-type biosensors, may be fabricated on one substrate 7, and the two lines of measurement signals, i.e., line 1 and line 2, as described above, may be acquired from each of the two measurement lines. The light emitted from one of the light sources 11 may be divided and further injected into each of the two measurement lines. Further, the light derived from each of the two measurement lines may be received by one of the photo detectors 12, e.g., the first photo detector 12A or the second photo detector 12B.

In the following description, the measurement chip with the symbol 1A refers to the measurement chip for line 1, the measurement chip with the symbol 1B refers to the measurement chip for line 2, and the measurement chip with the symbol 1 refers to the measurement chip encompassing both line 1 and line 2. The same meaning of the symbols A and B with respect to the measurement chip 1 (1A, 1B) applies to the light sources 11 (11 A, 11B) and the photo detectors 12 (12 A, 12B).

The measurement chip 1 encompassing both line 1 and line 2 may be placed at a predetermined location in the measuring device 10. Light emitted from the light source 11 may be introduced into the propagation layer 2 from the lower surface of the measurement chip 1 through the introductory part 3. Further, the light may get totally reflected inside the propagation layer 2 and may be derived out of the propagation layer 2 from the lower surface of the measurement chip 1 through the outgoing part 4 and is received by the photo detectors 12.

The light sources 11 having light source 11 A corresponding to line 1 and light source 11B corresponding to line 2 may emit visible light of a wavelength, for example, about 650 nm. The wavelength range of the light emitted by the light sources 11 may be in a range of, for example, 450 nm to 2000 nm. Preferably, the light emitted by the light sources 11 may be a Gaussian beam. A Gaussian beam is suitable for detecting changes in the light pattern (or intensity distribution) as rough shape of the light pattern may not change as the light propagates. In an example, the light emitted by the light source is a continuous wave. Although, the embodiments of the present disclosure describe the Gaussian beam to be Gaussian in the two dimensions of the X-axis direction and the Z-axis direction. However, this should not be construed as a limitation. In an example, the Gaussian beam may be Gaussian at least in the X-axis direction. For example, the light sources 11 may be a semiconductor laser based device that may be used to generate Gaussian beam. Further, the light source 11A for line 1 and the light source 11B for line 2 may emit light of a same wavelength.

Further, the photo detectors 12 (12 A, 12B) receive the light derived from the outgoing part 4. The photo detectors 12 may be composed of light receiving elements arranged in either one or two dimensions. Various image sensors such as a charge couple device (CCD) image sensor or a complementary metal oxide semiconductor (CMOS) image sensor may be used for the photo detectors 12.

In one embodiment, the control unit 13 is configured to analyze a change in a peak angle of the light received by the photodetectors 12. The control unit 13 may include an arithmetic unit (not shown in the figures) such as a CPU and a storage device (not shown in the figures) such as a memory, for example, a single board computer such as Raspberry Pi (R) or Arduino (R).

In one embodiment, the measuring unit 14 acquires intensity information of the light received by each light receiving element of the photodetectors 12. The acquired intensity information is transmitted to the control unit 13. The measuring unit 14 may be include a dedicated integrated circuit (IC).

Further, functions of the measuring device 10 are described with reference to FIG. 1-FIG. 3.

The light emitted from the light sources 11 may be further introduced into the introductory part 3 of the measurement chip 1. The introduced light further propagates while totally reflecting inside the propagation layer 2. An amount of phase shift when the light is totally reflected may depend on a magnitude of the refractive index of a surrounding material in contact with the propagation layer 2. As shown in FIG. 1(B), FIG. 1(C) and FIG. 2(B), there are regions on the surface 2A, such that an area in which the ligand layer 6 is in contact with the propagation layer 2, and the exposed area in which the coating layer 5 is in contact with the propagation layer 2. Further, the ligand layer 6 is formed on top of the coating layer 5. The amount of phase shift in total reflection of light depends on the refractive index within a penetration region of the evanescent light. Among the regions on the surface 2A of the propagation layer 2, a refractive index of the area in which the ligand layer 6 is in contact with the propagation layer 2 may be equal to the third refractive index of the ligand layer 6 (about 1.33), and a refractive index of the exposed area where the coating layer 5 is formed is the first refractive index of the coating layer (about 1.47). Therefore, the amount of phase shift on the surface of the propagation layer 2, when light is totally reflected, may be different for the area on the surface 2A where the ligand layer 6 is in contact with the propagation layer 2 and the exposed area where the coating layer 5 is in contact with the propagation layer 2.

Thus, the light propagating in the Y-axis direction between the introductory part 3 and the outgoing part 4 in the propagation layer 2 may change the phase distribution in the X-axis direction as the planar shape of the ligand layer 6 in a region formed on the surface 2A of the propagation layer 2 is such that the length of the region in the Y-axis direction in which the light propagates increases or decreases along the X-axis direction. Therefore, the phase distribution of the light derived from the outgoing part 4 may also be inclined along the X-axis direction and a propagating direction of the light may change. In the configuration shown in FIG. 2, the traveling direction or propagating direction of the light is inclined in the positive direction of the X-axis as the first refractive index of the coating layer 5 is higher than that the third refractive index of the ligand layer 6. Further, the propagating direction of the light may change, for example in the negative direction of the X-axis as the refractive index of the region 2A may increase based on the reaction between the ligand 82 and the analyte in the object (to be measured) and a part of the object to be measured is replaced by the analyte. In another embodiment, the third refractive index of the ligand layer 6 may also be increased due to the bulk effect or non-specific adsorption, further, reversing the light propagating direction in the negative direction of the X axis.

Therefore, the measuring device 10 may receive the light derived from the outgoing portion 4 at the far field (or through a Fourier transform lens) using the photodetectors 12 (the first photodetector 12 A and the second photodetector 12B) for each of the regions where the ligand layer 6 is formed (line 1) and the regions where the ligand layer 6 is not formed (line 2), respectively. Further, the measuring device 10 may measure a change in the angle at which the light intensity peaks, using the measuring unit 14. The change in the peak angle may be the same phenomenon as the change in the propagating direction of the light. The change in the angle at which the intensity peaks may correspond to the change in the propagating direction of the light. The change in the peak angle measured by the measuring unit 14 may be fed to the control unit 13 for recording the change appropriately in a storage device (memory) of the control unit 13. The control unit 13 may further include the arithmetic unit (CPU or central processing unit) that may subtract a graph of the change in the peak angle of line 2 from the graph of the change in the peak angle of line 1. To this end, the reaction between the ligand 82 in the ligand layer 6 and the analyte is confirmed based on a determination that the change in the subtracted graph is, for example, above a predetermined threshold. Alternatively, the control unit 13 may estimate the concentration or kinetic parameters of the analyte based on a shape of the subtracted graph. In this way, the control unit 13 may perform analytical processing to analyze changes in the light pattern. The control unit 13 may also perform analytical processing to analyze changes in the light propagating direction. In this way, the measuring device 10 may function as a measuring device to estimate the presence or absence of the analyte and the concentration or kinetic parameters of the analyte.

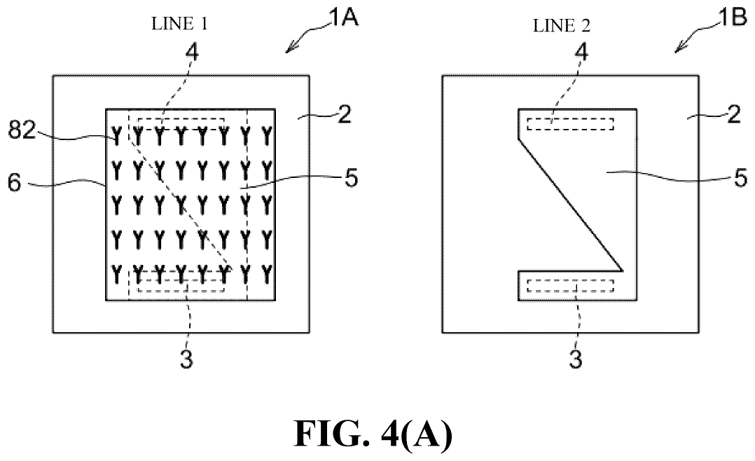

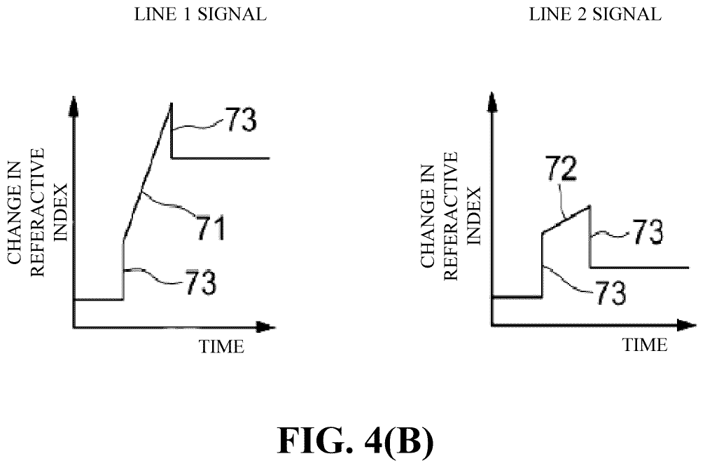

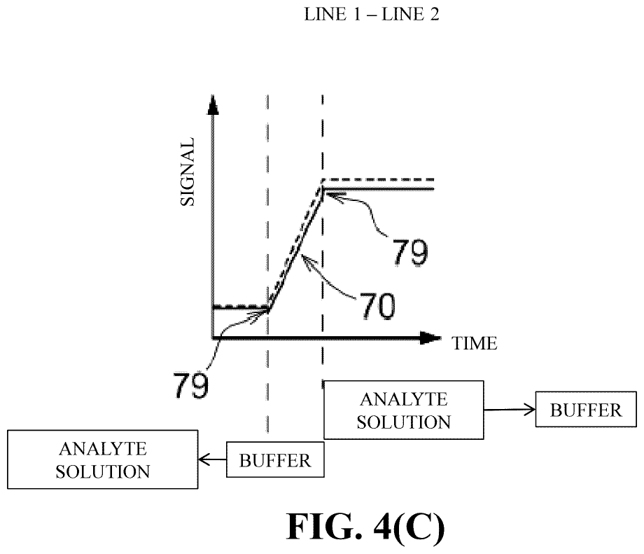

FIGS. 4(A)-4(C) are diagrams illustrating signals measured by the measurement chip 1, in accordance with an embodiment. As shown in FIG. 4(A), in the measurement chip 1A for the line 1, the ligand layer 6 may be formed uniformly on the surface 2A of the propagation layer 2 and the surface 5B of the coating layer 5. However, in the measurement chip 1B for the line 2, the ligand layer 6 may not be formed. FIG. 4(B) is a graph that schematically shows a change in the refractive indexes for each measurement signals of the line 1 and the line 2. FIG. 4(C) is a graph that schematically shows a difference between the measurement signals of the line 1 and the line 2 shown in FIG. 4(B). Referring to FIG. 4(B), the measurement signals from the line 1, where the ligand layer 6 is formed on the surface of the measurement chip 1A, contains information 71 on the desired reaction (For example, antigen-antibody reactions between the ligand 82 and the analyte) and non-specific adsorption, and information 73 on the bulk effect. Further, the measurement signal from line 2, where a ligand layer is not formed on the surface of the measurement chip 1B, contains information 72 on the non-specific adsorption only, and information 73 on the bulk effect.

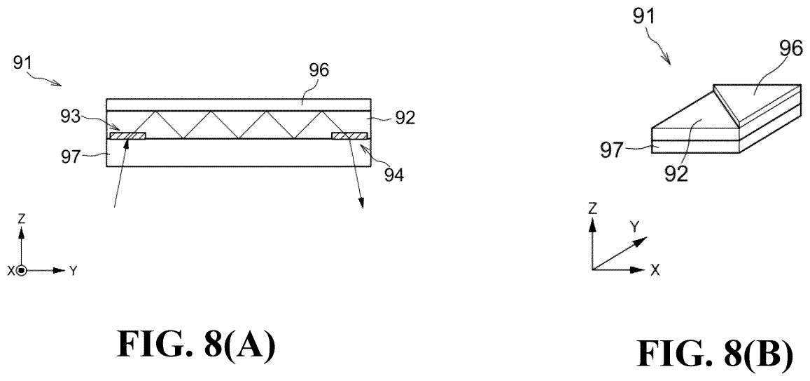

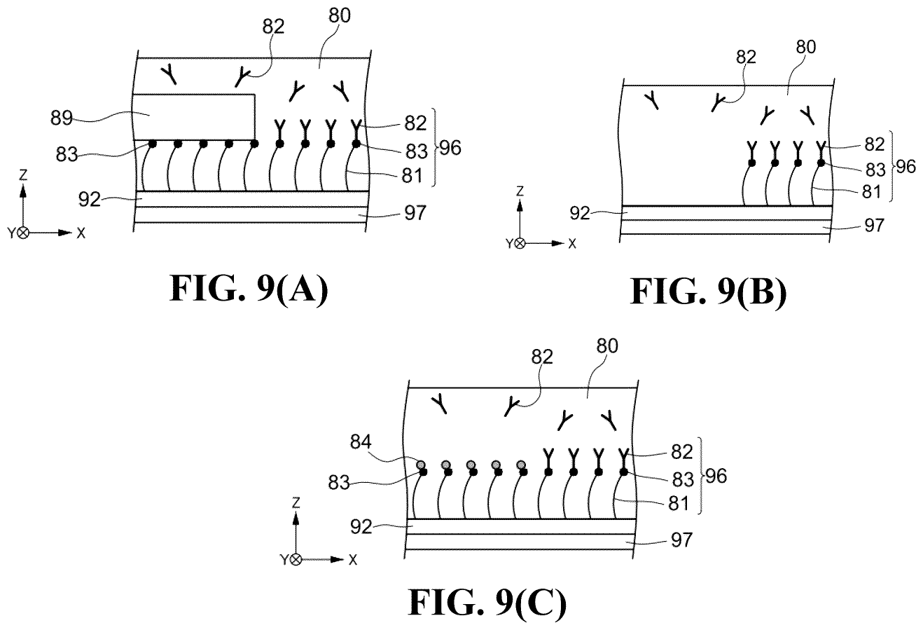

In an example, conventional measurement chips may only acquire signal corresponding to the difference signal shown in FIG. 4(C). However, when the effect due to the bulk effect shown by signal 73 or the effect due to the nonspecific adsorption shown by signal 72 is large, a component that cannot be canceled by subtracting the measurement signal may be generated, and such component that may not be canceled is a factor that reduces the reliability of measurement data in measurements using the conventional measurement chips. Details of the conventional measurement chips is described in conjunction with, for example, FIG. 8 and FIG. 9.

Referring to FIG. 4(C), the component shown by signal 79 may be the component that cannot be canceled due to the bulk effect, and the component that cannot be canceled due to the non-specific adsorption is included in the signal shown by 70 between the signal 79. The dashed line indicates the ideal analyte-ligand response. Thus, a magnitude of the effect caused by the bulk effect and a magnitude of the effect caused by the non-specific adsorption may not be obtained by the convention measurement chips, although it is desirable to obtain them as indicators for improving the reliability of the measurement data.

In an example, the measurement chip 1B corresponding to the line 2 may not detect the bulk effect and the non-specific adsorption on the surface 5A of the coating layer 5 of the measurement chip 1B. Thus, the measurement results reflect the bulk effect and the non-specific adsorption on the surface 2A of the propagation layer 2 in the exposed area exposed from the coating layer 5. Therefore, the measurement results reflecting the bulk effect and the non-specific adsorption may be obtained from the line 2 alone, and the measurement signal from the line 2 may be utilized as an indicator of the reliability of the measurement data.

On the other hand, the measurement results of the measurement chip 1A corresponding to the line 1 may, reflect the ligand-analyte reaction, bulk effects in the exposed area exposed from the coating layer 5, and non-specific adsorption based on the presence of the ligand layer 6 on the measurement chip 1A. According to one embodiment, as shown in FIG. 4(C), the difference signal between the measurement signals of the line 1 and the line 2 may be obtained, and the measurement chip 1 may function with further improved measurement reliability.

### Measurement Method

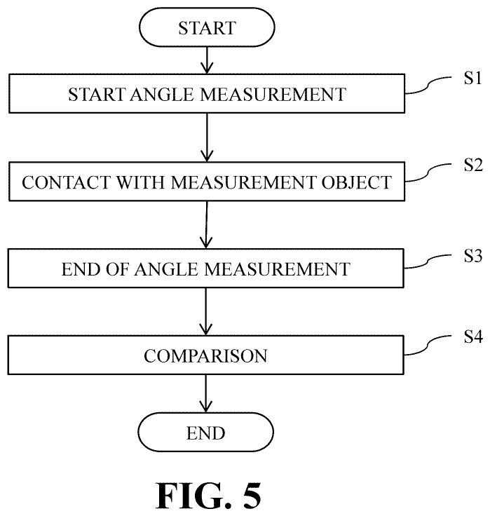

FIG. 5 is a flowchart illustrating a measurement method, in accordance with an embodiment. In an example, a measurement in the measurement method may be performed by totally reflecting light in the propagation layer 2 where the coating layer 5 is formed on the surface in a specific planar shape using the measurement chip 1.

For the measurement, two measurement chips corresponding to for the line 1 and the line 2 may be prepared. In the measurement chip 1A for the line 1, the ligand layer 6 is uniformly formed on the surface 2A of the propagation layer 2 and the surface 5A of the coating layer 5. In the measurement chip 1B for the line 2, no ligand layer is formed. Continuously, for each of the two measurement chips 1A and 1B, the base material 8 having the concave cross section, may be used to cover over an upper surface of the measurement chip 1, and the flow channel 9 may be provided between the upper surface of the measurement chip 1 and the base material 8. The two measurement chips 1A and 1B may be prepared in this way (e.g., the measurement chip 1A for the line 1 and the measurement chip 1B for the line 2), may be placed at the predetermined locations in the measuring device 10 as illustrated in FIG. 3. The following steps S1 to S3 are performed for each of the two measurement chips 1A and 1B for the line 1 and the line 2, respectively. Of the two measurement chips 1A and 1B for the line 1 and the line 2, the measurement chip 1A for the line 1 may have the ligand layer 6 formed, and the analyte in the measurement object solution may react with the ligand 82 in the ligand layer 6. The measurement chip 1B for the line 2 may not have a ligand layer formed, and the measurement chip 1B may be used for reference.

At step S1, a measurement of the propagating direction of the light may be started. Further, the propagating direction may be acquired and plotted in real time. On the surface of the propagation layer 2, the ligand layer 6 that reacts with the analyte in the object to be measured may be formed. The measurement of the propagating direction may be carried out by introducing light into the propagation layer 2 through the introductory part 3 of the measurement chip 1. Then, a peak position of the intensity of the light totally reflected in the propagation layer 2 may be measured using the photodetector 12. The light may then be derived out from the propagation layer 2 through the outgoing part 4. Here, an amount of change in the propagating or travelling direction (indicating peak angle) of the light approximately matches with a value obtained by dividing a distance between the measurement chip 1 and the photo detectors 12 from an amount of change in the peak position on the photo detectors 12.

At step S2, the measuring object is brought into contact with the measurement chip 1. A contact of the measuring object is made by bringing the measuring object containing the analyte into contact with the upper surface of the measurement chip 1. Generally, a buffer is brought into contact with the measurement chip 1 before and after the measuring object is brought into contact. The reason for bringing the buffer into contact with the measuring object before the measurement chips 1 is brought into contact with the measuring object is that if the measuring object is brought into contact with the measurement chip 1 without the buffer being brought into contact with it, the effects of a refractive index of the object solution itself and the effects such as a volume change of a scaffold material are reflected in the measurement signals. The buffer is brought into contact with the measuring object after the measuring object is brought into contact with the measurement chip 1 to improve an accuracy of subsequent analysis if the measurement signal of dissociation is also acquired.

In case of multiple measuring objects, the step S2 may be repeated multiple times. Further, regeneration is performed as an optional process when step S2 is repeated multiple times. The regeneration may be performed by exposing the measurement chip 1 to an acidic solution having a pH value of, for example, 3-1 to dissociate the ligand 82 and the analyte in a short time. Further, the regeneration may be omitted based on a determination that the analyte is rapidly dissociated.

At step S3, the measurement and plotting of the propagating direction of the light may be performed. Further, at step S4 (comparison step), a graph of a change of the peak angle acquired for the measurement chip 1A of the line 1 may be subtracted from a graph of a change of the peak angle acquired for the reference measurement chip 1B of the line 2 to obtain the graph depicted, for example, in FIG. 4(C). In this manner, the measurement from the reference measurement chip 1B is compared with the measurements of the measurement chip 1A.

With the graph of FIG. 4(C) obtained by subtracting, the reaction between the ligand 82 in the ligand layer 6 and the analyte (i.e., the analyte that is present in the object to be measured) may be confirmed. For example, when a change of the signal is above a predetermined threshold, the reaction may be ascertained. Alternatively, the concentration or kinetic parameters of the analyte may be estimated based on a curved shape of the signal in the FIG. 4(C). Thus, according to the measurement method in one embodiment, the presence or absence of the analyte may be determined or the concentration or kinetic parameters of the analyte may be estimated using the measurement chip 1.

Also, at step S4, from the measurement signal acquired for the reference chip 1B of the line 2, an amount of bulk effect and an amount of non-specific adsorption as described with reference to FIG. 4(C) may be estimated. In this way, the reliability of the measurement result may be evaluated using the measurement signal from the line 2.

In the measurement using the measurement chip 1, a number of times light is reflected inside the propagation layer 2 may be adjusted by changing a length of the measurement chip 1 in the Y-axis direction. Thus, the sensitivity of the measurement chip 1 may be changed. For example, longer the length of the measurement chip 1 in the Y-axis direction, greater the number of times the light may have reflected. Therefore, the sensitivity of the measurement chip 1 is improved.

In the measurement using the measurement chip 1, the amount of change in the peak angle may not change depending upon change in the output intensity of the light sources 11. This enables stable measurement using the measurement chip 1, even if the operation of the light sources 11 is somewhat unstable.

### Other Forms

While the present disclosure has been described above in terms of specific embodiment(s), the present disclosure is not limited to the above-mentioned embodiment(s).

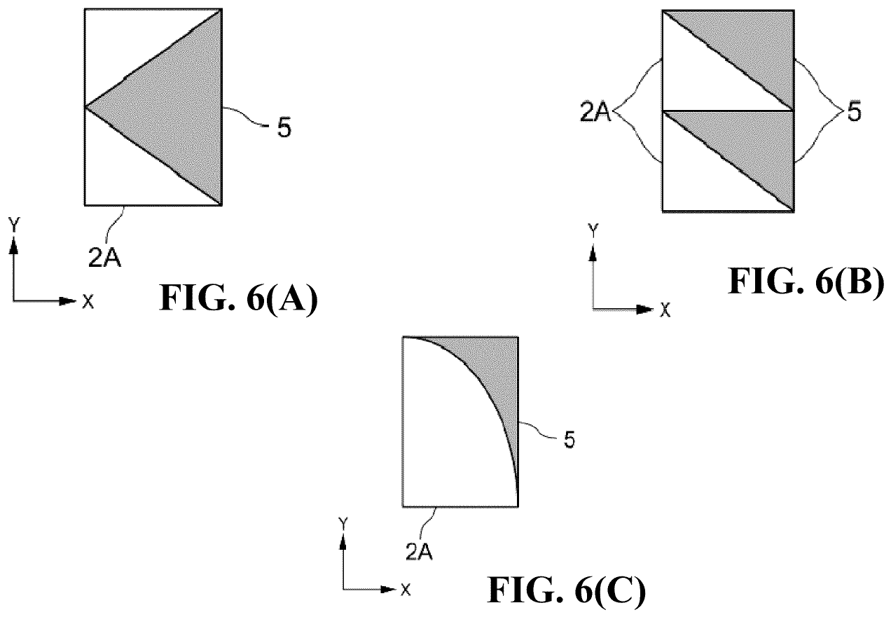

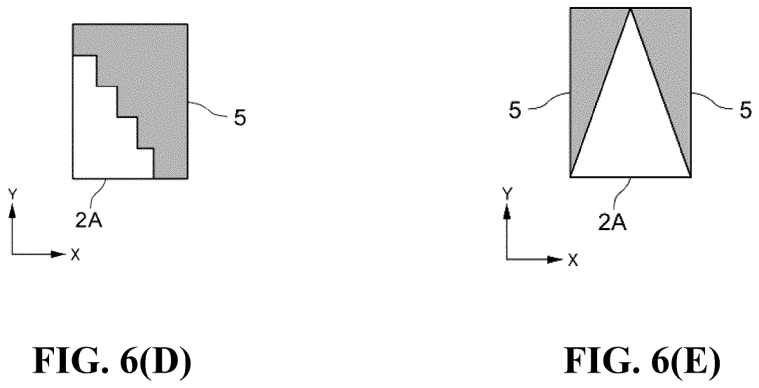

In the above embodiment(s), as illustrated in FIGS. 1(A)-1(C) and FIGS. 2(A) and 2(B), the planar shape of the coating layer 5 is a right-angled triangle, and the planar shape of the ligand layer 6 in the portion formed on the surface 2A of the propagation layer 2 in the area exposed from the coating layer 5 is also a right-angled triangle. However, the planar shape of the coating layer 5 is not limited to the exemplified right-angled triangle, and the planar shape of the ligand layer 6 in the portion formed on the surface 2A of the propagation layer 2 in the area exposed from the coating layer 5 is not limited to the exemplified right-angled triangle. As illustrated in FIG. 6, the planar shape of the coating layer 5 may include a shape in which the length of the light propagation direction (Y-axis direction) increases or decreases along the direction perpendicular to the propagation direction (X-axis direction). That is, the length in the formed region of the coating may include a shape in which the length of the propagating direction (Y-axis direction) increases or decreases along the direction perpendicular to the propagation direction (X-axis direction). The increasing or decreasing part may be included as part of a particular planar shape or may increase or decrease throughout the particular planar shape. Thus, the planar shape of the ligand layer 6 in the part formed on the surface 2A of the propagation layer 2 in the area exposed from the coating layer 5 may also include a shape in which the length of the light propagating direction (Y-axis direction) increases or decreases along the direction perpendicular to the propagation direction (X-axis direction).

FIGS. 6(A)-6(E) are diagrams showing variations in the planar shape of the coating layer 5 in the measurement chip 1, in accordance with an embodiment. A planar shape of the coating layer 5 illustrated in FIG. 6(A) may be an isosceles triangle, and the length of the propagating direction (the Y-axis direction) may be extended continuously and linearly along the direction perpendicular to the light propagating direction (the X-axis direction). In the example, the ligand layer 6 is uniformly formed on the surface 2A. The planar shape of the ligand layer 6 in the portion formed on the surface 2A of the propagation layer 2 in the area exposed from the coating layer 5 becomes a shape in which two right-angled triangles facing each other, and the ligand content in the light propagation direction changes continuously and linearly along a vertical direction.

The planar shape of the coating layer 5, as illustrated in FIG. 6(B) may be a shape in which two right-angled triangles stand side by side, and the length of the light propagating direction (the Y-axis direction) may become continuously and linearly longer along the direction perpendicular to the light propagating direction (the X-axis direction). In an example, the ligand layer 6 is uniformly formed on the surface 2A. The planar shape of the ligand layer 6 in the portion formed on the surface 2A of the propagation layer 2 in the area exposed from the coating layer 5 may also become a shape in which two right-angled triangles stand side by side, and the ligand content in the light propagation direction may change continuously and linearly along the vertical direction.

In a mode illustrated, for example, in the FIG. 6(A) and the FIG. 6(B), since the phase distribution of light derived from the outgoing part 4 is similar to the mode illustrated, for example, in FIG. 1 and FIG. 2, an effect similar to the mode illustrated in FIGS. 1(A)-1(C) and FIG. 2(A) and FIG. 2(B) may be expected. Here, in the mode illustrated in FIG. 6(A), the amount of change in the peak angle with respect to the refractive index change increases more than in the mode illustrated, for example, in FIGS. 1(A)-1(C), which is advantageous against noise such as, vibration. In the mode illustrated in FIG. 6(A), the width in the X-axis direction of the flow channel 9 illustrated in FIG. 2(A) and FIG. 2(B) may be narrowed based on a determination that the propagation process of propagating light and the extent of existence on the X-axis in the outgoing part 4 become smaller than in the case illustrated in FIGS. 1(A)-1(C), that further enables more precise measurement. Furthermore, the propagation process of propagating light and the existence range on the X-axis in the outgoing part 4 can be made smaller, and the width of the flow channel 9 in the X-axis direction can be made narrower by tilting the incident direction of the light source in the negative direction of the X-axis in the FIG. 6(A). Note that a similar effect can be obtained when the arrangement of the coating layer 5 in the FIG. 6(A) is switched with the surface 2A of the propagation layer 2 in the area exposed from the coating layer 5.

More specifically, there are, for example, following three indicators (referred to as, a first indicator, a second indicator and a third indicator) as indicators of the performance of the measurement chip 1. According to the mode illustrated in FIG. 6(A), the first indicator and the third indicator may be improved.

The first indicator may indicate an amount of angular change/refractive index change. For example, larger amount of angular change indicates more sensitive due to, for example, angular change noise such as vibration, vs.

The second indicator may indicate angular change/beam width and refractive index change. For example, larger angular change indicates more sensitive due to, for example, intensity change noise such as electrical noise.

The third indicator may indicate narrowness of the beam's range in the waveguide. For example, narrower the channel, the more accurate the measurement.

In the planar shape of the coating layer 5 shown, for example, in FIG. 6(C), the length of the light propagating direction (Y-axis direction) may be continuously and non-linearly lengthened along the direction perpendicular to the light propagating direction (X-axis direction). In the example of FIG. 6(C), when the ligand layer 6 is uniformly formed on the surface, the ligand content changes continuously and non-linearly along the vertical direction in the part of the ligand layer 6 formed on the surface 2A of the propagation layer 2 in the area exposed from the coating layer 5. In this case, the spreading angle of the light changes as the propagating direction of the light changes. Therefore, elements other than the propagating direction of the light may also change.

The planar shape of the coating layer 5 illustrated in the FIG. 6(D) may be a stepped shape, and the length of the light propagating direction (Y-axis direction) changes discontinuously along the direction perpendicular to the light propagating direction (X-axis direction). In this case, diffracted light appears, and the traveling direction and intensity ratio of each order of diffracted light may change.

The examples shown in FIGS. 1(A)-1(C), FIG. 2A and FIG. 2(B), FIG. 6(A), FIG. 6(B), FIG. 6(C), and FIG. 6(D) are examples where the density of the ligand layer 6 is constant and the length of the light propagating direction (Y-axis direction) changes monotonically along the vertical direction. The examples shown in FIGS. 1(A)-1(C), FIG. 2A and FIG. 2(B), FIG. 6(A), FIG. 6(B), and FIG. 6(C), are examples where the density of the ligand layer is constant and the length of the light propagation direction (Y-axis direction) changes continuously along the vertical direction. The examples shown in FIGS. 1(A)-1(C), FIG. 2A and FIG. 2(B), FIG. 6(A), and FIG. 6(B) are examples where the density of the ligand layer is constant and the length of the light propagation direction (Y-axis direction) changes linearly along the vertical direction.

The planar shape of the coating layer 5 shown in FIG. 6(E) may be outside the isosceles triangle and the length of the light propagation direction (Y-axis direction) decreases continuously and linearly along the direction perpendicular to the light propagation direction (X-axis direction) and then increases continuously and linearly. Since the refractive index of the coating layer 5 is usually sufficiently larger than that of the ligand layer 6, in this case, the light derived from the outgoing part 4 may be divided into two pieces and the difference between the peak angles of the two pieces of light may be obtained, which has the advantage of canceling the noise caused by vibration and the like.

It should be noted that various planar shapes of the coating layer 5 exemplified in FIGS. 1(A)-1(C), FIG. 2A and FIG. 2(B), and FIG. 6(A)-6(E),) may be reversed along the X axis, further reversed along the Y axis, and furthermore reversed between the region of the coating layer 5 and the region of the propagation layer 2, and a combination thereof, and similar effects may further be obtained in these planar shapes.

In the above embodiment, examples of antigens and antibodies are shown as combinations of analytes and ligands, but combinations are not limited to this. The combinations of analytes and ligands may also include enzymes and substrates, hormones and receptors, and DNA (deoxyribonucleic acid) complements. Even in these cases, on the surface 2A of the propagation layer 2, an amount of phase shift in total reflection of light differs between the surface 2A of the propagation layer 2 in the exposed area exposed from the coating layer 5 and the area where the coating layer 5 is formed, and the amount of phase shift in the area of the surface 2A of the propagation layer 2 in the exposed area exposed from the coating layer 5 may be changed by the combination of analytes and ligands.

In the measurement device 10 and the measurement method using the measurement chip 1 in the above embodiment, the binding reaction of biomolecules is used as an example, but it is possible to apply it if the reaction involves a change in refractive index even if it is not the binding reaction of the biomolecules to be exemplified. As an example, the measurement device 10 and the measurement method using the measurement chip 1 in the above embodiment may be applied to gas sensors, etc. In this case, the gas may be used as an analyte, and a chemical substance whose refractive index changes when it reacts with the gas may be used as a ligand.

### EXAMPLE

Examples of the present disclosure are given below to further clarify the features of the explained in the present disclosure.

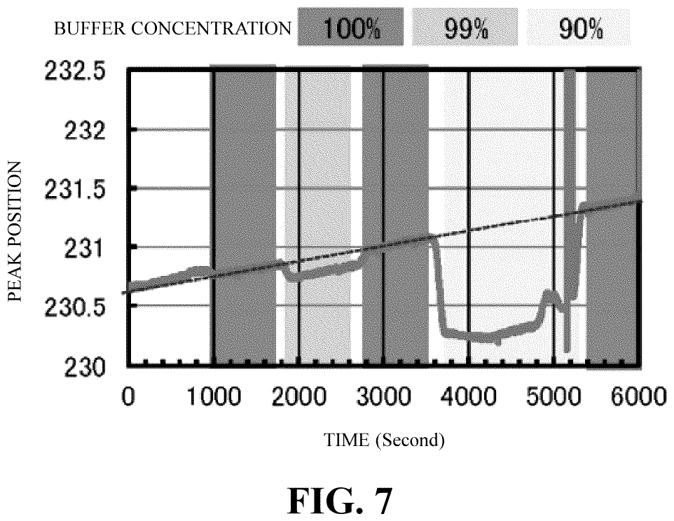

In an example, a performance of a fabricated measurement chip based on the measurement chip 1, is evaluated. The performance is evaluated by changing a salt concentration of a buffer solution flowing over a top surface of the measurement chip 1 and using the measuring device 10 to observe the change in the peak position of the light received by the receiver or the photodetector 12. A graph of the observation results is shown in FIG. 7.

FIG. 7 illustrates a graph showing results of a performance evaluation of the measurement chip 1, according to an embodiment. The peak position of the observed light may be changed at multiple timings (for example, about 1,700 seconds, about 2,700 seconds, and about 3,600 seconds) based on a determination that when the salt concentration in the buffer solution is changed. This may confirm that the bulk effect, which is the effect of the refractive index of the buffer solution, may be observed in the fabricated measurement chip 1.

In another example, a relationship between sensitivity of the refractive index at the surface 5A of the coating layer 5 and the thickness of the coating layer 5 is numerically simulated. The numerical simulations is carried out with an assumption that silicon dioxide (SiO₂) is used as the coating layer 5 for the light having wavelength of about 520 nm and the light having wavelength of about 810 nm that is totally reflecting the propagation layer 2, respectively. For the light incident on the introduction of the measurement chip 1, the light with the electric field in the Z-axis direction, as shown in FIG. 1(A), is defined as P-polarized light, and the light with the electric field in the X-axis direction is defined as S-polarized light. It should be noted that the use of P-polarized light for the incident light is more sensitive and practical as the coupling efficiency to the propagation layer at the introduction is higher. When P-polarized light is used, the optimum propagation layer thickness is slightly less than twice that of S-polarized light. Conditions and results of the numerical simulations are shown, for example, in tables 1 and table 2.

TABLE 1

| | Wave- | Wave- | Wave- | Wave- |
|---|---|---|---|---|
| | length | length | length | length |
| | 520 nm S | 520 nm P | 810 nm S | 810 nm P |
| | Polariza- | Polariza- | Polariza- | Polariza- |
| | tion | tion | tion | tion |
| Propagation Layer | 55 nm | 100 nm | 95 nm | 165 nm |
| Thickness | | | | |
| Propagation Layer | 2.037 | 2.037 | 1.987 | 1.987 |
| Refractive Index | | | | |
| SiO2 Refractive | 1.46 | 1.46 | 1.45 | 1.45 |
| Index | | | | |

TABLE 2

| SiO2 | Wavelength | Wavelength | Wavelength | Wavelength |
|---|---|---|---|---|
| Thick- | 520 nm | 520 nm | 810 nm | 810 nm |
| ness | S Polariza- | P Polariza- | S Polariza- | P Polariza- |
| (nm) | tion (%) | tion (%) | tion (%) | tion (%) |
| 0 | 100 | 100 | 100 | 100 |
| 100 | 16.8 | 18 | 31.6 | 34.1 |
| 200 | 3.06 | 2.98 | 10.4 | 10.9 |
| 250 | 1.32 | 1.21 | 6.02 | 6.16 |
| 300 | 0.573 | 0.492 | 3.5 | 3.47 |
| 400 | 0.107 | 0.0805 | 1.19 | 1.1 |
| 500 | 0.02 | 0.013 | 0.405 | 0.349 |

Based on a conclusion drawn from the table 1 and table 2, when the coating layer of SiO2 is thick, the evanescent light penetrating from the inside of the propagation layer 2 toward the ligand layer 6 on the surface of the coating layer 5 is more attenuated. As a result, the less sensitive the refractive index change is on the surface of the coating layer, which is preferable. For practical use, a refractive index sensitivity of about 1% is sufficient. Based on the numerical simulation results shown in the Table 2, it is shown that the thickness of the coating layer SiO2 is preferably about 250 nm or more for a wavelength of 520 nm and about 400 nm or more for a wavelength of 810 nm.

FIG. 8(A) and 8(B) are diagrams of the schematic structure of the measurement chip 91 in Patent Document 1, in accordance with an embodiment. As shown in a side view of FIG. 8(A) and a perspective view of FIG. 8(B), the measurement chip 91 in Patent Document 1 is equipped with a propagation layer 92 where light propagates, an introductory part 93 which introduces light into the propagation layer 92, an outgoing part 94 which derives light from the propagation layer 92, and a ligand layer 96, and the region of the ligand layer 96 is formed in a specific planar shape on the surface of the propagation layer 92. Specifically, as shown in FIG. 8(B), the region of the ligand layer 96 is formed in a planar shape (e.g., right-angled triangles) such that the length of the ligand layer 96 in the propagation direction of light (Y-axis direction in the figure) increases or decreases along the direction perpendicular to the propagation direction (X-axis direction in the figure). The transparent substrate 97 provided on the lower surface of the propagation layer 92 is of an arbitrary configuration.

FIG. 9(A)-9(C) are diagrams showing three methods of forming a ligand layer in a specific planar shape in the measurement chip in Patent Document 1, in accordance with an embodiment. There are three methods for forming the ligand layer 96 in such a specific planar shape, for example, as shown in FIG. 9. The first method is to uniformly form the scaffold material 81 on the surface of the propagation layer 92 and then pattern the ligand 82 into the specific planar shape described above using a mask 89 and a ligand solution 80, as shown in FIG. 9(A). The second method is to pattern the scaffold material 81 into the specific planar shape described above using a mask 89 when forming the scaffold material 81 on the surface of the propagation layer 92. As shown in FIG. 9(B), the scaffold material 81 is pre-patterned into the specific planar shape described above, so when the scaffold material 81 is brought into contact with the ligand solution 80, the ligand 82 is formed into this specific planar shape. The third method is that after the scaffold material 81 is uniformly formed on the surface of the propagation layer 92, the binding site 83 of the scaffold material 81 in the region having the specific planar shape is pre-blocked using the mask 89 and blocking solution. As shown in FIG. 9(C), the binding site 83 of the scaffold material 81 in the region having the specific planar shape is left in a state 84 in which it may not bind to the ligand 82.

Since all three methods of forming the ligand layer 96 in the specific planar shape are wet processes, the reproducibility of the process is not high and the cost is high. In forming the ligand layer 96 in the specific planar shape mentioned above, further improvement of the process reproducibility and cost reduction are required. As process reproducibility improves, measurement reproducibility and measurement accuracy also improve.

In addition, the magnitude of the effect due to the refractive index of the object to be measured (also called the bulk effect) and the magnitude of the effect due to nonspecific adsorption is known to be indicators of the reliability of measurement results in optical waveguide type measurement chips. However, in the measurement chip 91 in Patent Document 1, it is not possible to directly measure the magnitude of the effect due to these bulk effects or the magnitude of the effect due to nonspecific adsorption. To improve the reliability of measurement results, it is required to directly measure the magnitude of the effect due to these bulk effects or the magnitude of the effect due to nonspecific adsorption.

Embodiments of the present disclosure, as described in conjunction with FIGS. 1-7 aim to overcome the aforementioned drawbacks associated with the conventional optical waveguide-type measurement chips. The present disclosure provides measurement chips that enable accurate and reliable measurement of presence or absence, concentration and/or kinematics of an analyte using a coating layer that increases or decreases the length of the propagating direction in a formed region of a coating, along a direction perpendicular to the propagating direction.

The above description describes the embodiments of the present disclosure. Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

It is to be understood that not necessarily all objectives or advantages may be achieved in accordance with any particular embodiment described herein. Thus, for example, those skilled in the art will appreciate that certain embodiments may be configured to operate in a manner that achieves or optimizes one advantage or group of advantages as taught herein without necessarily achieving other objects or advantages as may be taught or suggested herein.

All processes described herein may be embodied in, and fully automated via, software code modules executed by a computing system that includes one or more computers or processors. The software code modules may be stored in any type of non-transitory computer-readable medium or other computer storage device. Some or all methods may be embodied in specialized computer hardware.

Many other variations other than those described herein will be apparent from this disclosure. For example, depending on the embodiment, certain actions, events, or functions of any of the algorithms described herein may be performed in different sequences, and may be added, merged, or excluded altogether (e.g., not all described actions or events are required to execute the algorithm). Moreover, in certain embodiments, operations or events are performed in parallel, for example, through multithreading, interrupt handling, or through multiple processors or processor cores, or on other parallel architectures, rather than sequentially. In addition, different tasks or processes can be performed by different machines and/or computing systems that can work together.

The various exemplary logical blocks and modules described in connection with the embodiments disclosed herein can be implemented or executed by a machine such as a processor. The processor may be a microprocessor, but alternatively, the processor may be a controller, a microcontroller, or a state machine, or a combination thereof. The processor can include an electrical circuit configured to process computer executable instructions. In another embodiment, the processor includes an application specific integrated circuit (ASIC), a field programmable gate array (FPGA), or other programmable device that performs logical operations without processing computer executable instructions. The processor can also be implemented as a combination of computing devices, e.g., a combination of a digital signal processor (DSP) and a microprocessor, a plurality of microprocessors, one or more microprocessors in conjunction with a DSP core, or any other such configuration. Although described herein primarily with respect to digital technology, the processor may also include primarily analog components. For example, some or all of the signal processing algorithms described herein may be implemented by analog circuitry or mixed analog and digital circuitry. A computing environment may include any type of computer system, including, but not limited to, a computer system that is based on a microprocessor, mainframe computer, a digital signal processor, a portable computing device, a device controller, or a computing engine within the device.

Unless otherwise stated, conditional languages such as “can,” “could,” “will,” “might,” or “may” are understood within the context as used in general to convey that certain embodiments include, while other embodiments do not include, certain features, elements and/or steps. Thus, such conditional languages are not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Disjunctive languages, such as the phrase “at least one of X, Y, or Z,” unless specifically stated otherwise, is understood with the context as used in general to present that an item, term, etc., may be either X, Y, or Z, or any combination thereof (e.g., X, Y, and/or Z). Thus, such a disjunctive language is not generally intended to, and should not, imply that certain embodiments require at least one of X, at least one of Y, or at least one of Z to each be present.

Any process descriptions, elements, or blocks in the flow diagrams described herein and/or shown in the accompanying drawings should be understood as potentially representing modules, segments, or parts of code, including one or more executable instructions for implementing a particular logical function or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C. The same holds true for the use of definite articles used to introduce embodiment recitations. In addition, even if a specific number of an introduced embodiment recitation is explicitly recited, those skilled in the art will recognize that such recitation should typically be interpreted to mean at least the recited number (e.g., the bare recitation of “two recitations,” without other modifiers, typically means at least two recitations, or two or more recitations).

It will be understood by those within the art that, in general, terms used herein, are generally intended as “open” terms (e.g., the term “including” should be interpreted as “including but not limited to,” the term “having” should be interpreted as “having at least,” the term “includes” should be interpreted as “includes but is not limited to,” etc.).

For expository purposes, the term “horizontal” as used herein is defined as a plane parallel to the plane or surface of the floor of the area in which the system being described is used or the method being described is performed, regardless of its orientation. The term “floor” can be interchanged with the term “ground” or “water surface”. The term “vertical” refers to a direction perpendicular to the horizontal as just defined. Terms such as “above,” “below,” “bottom,” “top,” “side,” “higher,” “lower,” “upper,” “over,” and “under” are defined with respect to the horizontal plane.

As used herein, the terms “attached,” “connected,” “coupled,” and other such relational terms should be construed, unless otherwise noted, to include removable, moveable, fixed, adjustable, and/or releasable connections or attachments. The connections/attachments can include direct connections and/or connections having intermediate structure between the two components discussed.

Numbers preceded by a term such as “approximately,” “about,” and “substantially” as used herein include the recited numbers, and also represent an amount close to the stated amount that still performs a desired function or achieves a desired result. For example, the terms “approximately,” “about,” and “substantially” may refer to an amount that is within less than 10% of the stated amount. Features of embodiments disclosed herein preceded by a term such as “approximately,” “about,” and “substantially” as used herein represent the feature with some variability that still performs a desired function or achieves a desired result for that feature.

It should be emphasized that many variations and modifications may be made to the above-described embodiments, the elements of which are to be understood as being among other acceptable examples. All such modifications and variations are intended to be included herein within the scope of this disclosure and protected by the following claims.

What is claimed is:

1. A measurement chip, comprising:
a propagation layer configured to allow light to propagate in a propagating direction;
an introductory part configured to introduce the light into the propagation layer;
an outgoing part configured to outgo the light from the propagation layer;
a coating layer configured to be formed on a surface of the propagation layer, the coating layer is further configured to increase or decrease a length of the propagating direction in a formed region of a coating, along a direction perpendicular to the propagating direction; and
a ligand configured to react with an analyte on the surface of the propagation layer at least in an exposed area exposed from the coating layer.

2. The measurement chip as claimed in claim 1, further comprising:
a ligand layer formed by modifying the ligand on the surface of the propagation layer in the exposed area.

3. The measurement chip as claimed in claim 2, wherein:
a first refractive index of the coating layer is smaller than a second refractive index of the propagation layer and greater than a third refractive index of the ligand layer.

4. The measurement chip as claimed in claim 1, wherein:
the coating layer is configured to be formed between the introductory part and the outgoing part of the measurement chip.

5. The measurement chip as claimed in claim 1, wherein:
the coating layer is configured to be formed on at least one of: the introductory part, or the outgoing part of the measurement chip.

6. The measurement chip as claimed in claim 1, wherein:
the coating layer is further configured to continuously increase or decrease the length of the formed region of the coating in the propagating direction along the direction perpendicular to the propagating direction.

7. The measurement chip as claimed in claim 1, wherein:
the coating layer is further configured to linearly increase or decrease the length of the formed region of the coating in the propagating direction, along the direction perpendicular to the propagating direction.

8. The measurement chip as claimed in claim 1, wherein:
the coating layer has a thickness equal to or greater than a decay length of evanescent light penetrating from the surface of the propagation layer towards a medium on a side of the coating layer.

9. The measurement chip as claimed in claim 1, wherein:
the coating layer is formed using silicon dioxide.

10. The measurement chip as claimed in claim 1, wherein:
the coating layer is formed using a mixture of silicon dioxide and a metal oxide.

11. The measurement chip as claimed in claim 1, wherein:
the coating layer is formed using a mixture of silicon dioxide and aluminum oxide (Al₂O₃).

12. The measurement chip as claimed in claim 1, further comprising:
a characteristic adjustment film arranged on a surface of the coating layer.

13. The measurement chip as claimed in claim 12, wherein:
the characteristic adjustment film is formed using a metal oxide.

14. The measurement chip as claimed in claim 1, wherein:
a phase distribution of the light changes due to a change in a refractive index around the propagation layer caused by a reaction between the analyte with the ligand.

15. A measuring device:
a measurement chip comprising:
a propagation layer configured to allow light to propagate in a propagating direction,
an introductory part configured to introduce the light into the propagation layer;
an outgoing part configured to outgo the light from the propagation layer;
a coating layer configured to be formed on a surface of the propagation layer, the coating layer is further configured to increase or decrease a length of the propagating direction in a formed region of a coating, along a direction perpendicular to the propagating direction; and
a ligand configured to react with an analyte on the surface of the propagation layer at least in an exposed area exposed from the coating layer; and
a light source configured to introduce the light to the introductory part of the measurement chip;
a photodetector configured to receive the light outgone from the outgoing part of the measurement chip; and
a processing circuitry configured to analyze a change in a pattern of the light received by the photodetector that changes based on the reaction between the analyte and the ligand of the measurement chip.

16. The measuring device as claimed in claim 15, wherein:
the processing circuitry is further configured to analyze a change in the propagating direction of the light.

17. A measurement method, comprising:
introducing light into a propagation layer;
causing the light to totally reflect in the propagation layer having a surface on which a ligand reacting to an analyte is formed in an exposed area from a coating layer formed on the surface of the propagation layer; and
deriving the light from the propagation layer, wherein a length of the propagating direction in a formed region of a coating of the coating layer increases or decreases along a direction perpendicular to the propagating direction.

18. The measurement method as claimed in claim 17, further comprising:
analyzing a change in a pattern of the light derived from the propagation layer, wherein:
the change in the pattern is caused due to a reaction between the analyte and the ligand of the measurement chip.

19. The measurement method as claimed in claim 17, further comprising:
analyzing a change in a traveling direction of the light derived from the propagation layer.

20. The measurement method as claimed in claim 17, wherein:
a first refractive index of the coating layer is smaller than a second refractive index of the propagation layer and greater than a third refractive index of a ligand layer that is formed by modifying the surface of the propagation layer in the exposed area using the ligand.
